(19) United States
(12) Patent Application Publication
(10) Pub. No.: US 2024/0310164 A1
(43) Pub. Date: Sep. 19, 2024

(54) DEVICES, SYSTEMS, AND METHODS FOR TOPOGRAPHIC ANALYSIS OF A BIOLOGICAL SURFACE

(71) Applicant: Linus Biotechnology Inc., New Rochelle, NY (US)

(72) Inventors: Manish ARORA, New Rochelle, NY (US); Paul CURTIN, Jersey City, NJ (US); Christine AUSTIN, New York, NY (US)

(21) Appl. No.: 18/672,412

(22) Filed: May 23, 2024

Related U.S. Application Data
(63) Continuation of application No. PCT/US2022/080446, filed on Nov. 23, 2022.
(60) Provisional application No. 63/283,139, filed on Nov. 24, 2021.

(51) Int. Cl. G01B 11/24; G01N 1/36

(52) U.S. Cl. CPC G01B 11/24; G01N 1/36

(57) ABSTRACT

Provided are herein are devices, systems, and methods of measuring the topography of a biological surface and deriving a probabilistic prediction of the presence or lack thereof phenotypic feature.

### CROSS-REFERENCE

This application is the by-pass continuation application of International Application No. PCT/US2022/080446, filed Nov. 23, 2022, which claims the benefit of U.S. Provisional Application No. 63/283,139 filed Nov. 24, 2021, each of which are incorporated herein by reference in their entirety.

### BACKGROUND

Non-invasive diagnostic technologies have drastically advanced over the last decade enabling one to capture rich representative datasets of complex biological and physiologic systems. Such technologies have appropriately innovated in improving signal to noise and molecular specificity, but are still merely a snapshot at a given time. Diagnostic platforms are at a developmental inflection point whereby integration of predictive models, machine learning or artificial intelligence integration is necessary to leverage interpret the rich datasets beyond what a human may be able to gamer to provide insightful and actionable predictions. For this reason, there is a clinical unmet need of innovations in diagnostic platforms to leverage such a rich dataset to provide paradigm shifting predictions of a biological or physiological states.

### SUMMARY

Aspects of the disclosure provided herein comprise a method for determining the topography of a surface of a biological sample, comprising: (a) receiving a biological sample; (b) mounting the biological sample into a fixture; (c) contacting the biological sample with a compressible body optically coupled to at least one light source and at least one sensor, wherein the at least one light source generates a multi-dimensional dataset of the surface of the biological sample detected by the at least one sensor; and (d) determining a surface profile of the biological sample from the multi-dimensional dataset. In some embodiments, the biological sample comprises an ex-vivo or in-vivo tooth. In some embodiments, the compressible body comprises a gel cartridge. In some embodiments, the fixture comprises putty, a mechanical mount, adhesive compound, or any combination thereof. In some embodiments, the method further comprises calibrating the compressible body on standard glass, ball grid array, groove targets, or any combination thereof. In some embodiments, the at least one light source comprises at least one coherent laser, incoherent laser, pulsed laser, light emitting diode, super-luminescent diode, or any combination thereof. In some embodiments, the method further comprises cleaning the biological sample with water. In some embodiments, the compressible body spatial position is releasably lockable. In some embodiments, the at least one light source and the at least one sensor are electrically in communication with a processor via a button, switch, trigger, or any combination thereof configured to initiate illumination of the biological sample and detection of the multi-dimensional dataset of the surface. In some embodiments, the multi-dimensional dataset comprises at least 1 dimension. In some embodiments, the surface profile is measured from the incisal edge to the cervical end of the crown of the ex-vivo or in-vivo tooth.

Aspects of the disclosure provided herein comprise a method for determining the topography of a surface of a biological sample, comprising: (a) receiving a biological sample; (b) mounting the biological sample into a fixture; (c) illuminating the biological sample with at least one light source and at least one sensor, wherein the at least one light source generates a multi-dimensional dataset of the surface of the biological sample detected by the at least one sensor; and (d) determining a surface profile of the biological sample from the multi-dimensional dataset. In some embodiments, the biological sample comprises ex-vivo or in-vivo tooth. In some embodiments, the fixture comprises putty, a mechanical mount, stabilizer, adhesive compound, or any combination thereof. In some embodiments, the at least one light source comprises at least one coherent laser, incoherent laser, pulsed laser, light emitting diode, super-luminescent diode, or any combination thereof. In some embodiments, the method further comprises cleaning the biological sample with water. In some embodiments, the at least one light source and the at least one sensor are electrically in communication with a processor via a button, switch, trigger, or any combination thereof configured to initiate illumination of the biological sample and detection of the multi-dimensional dataset of the surface. In some embodiments, the multi-dimensional dataset comprises at least 1 dimension. In some embodiments, the surface profile is measured from the incisal edge to the cervical end of the crown of the ex-vivo or in-vivo tooth.

Aspects of the disclosure provided herein comprise a system for determining the topography of a surface of a biological sample, comprising: (a) a fixture mechanically configured to constrain a three-dimensional position of a biological sample; (b) a compressible body optically coupled to at least one light source and at least one sensor, wherein the at least one light source generates a multi-dimensional dataset of a surface of the biological sample detected by the at least one sensor; and (c) a processor electrically coupled to the at least one light source and the at least one sensor, wherein the processor comprises a set of programmed instructions stored on a non-transitory storage medium configured to cause the processor to determine a topography of the surface of the biological sample from the multi-dimensional dataset. In some embodiments, the biological sample comprises a tooth. In some embodiments, the compressible body comprises a gel cartridge. In some embodiments, the fixture comprises putty, a mechanical mount, adhesive compound, or any combination thereof. In some embodiments, the at least one light source comprises at least one coherent laser, incoherent laser, pulsed laser, light emitting diode, super-luminescent diode, or any combination thereof. In some embodiments, the compressible body spatial position is releasably lockable. In some embodiments, the at least one light source and the at least one sensor are electrically in communication with a processor via a button, switch, trigger, or any combination thereof configured to initiate illumination of the biological sample and detection of the multi-dimensional dataset of the surface. In some embodiments, the system further comprises a rigid guide member mechanically coupled to the compressible body configured to position the compressible body in relation to the biological sample. In some embodiments, the compressible body is housed in a locking mechanical member configured to fix the three-dimensional position of the compressible body. In some embodiments, the multi-dimensional dataset comprises at least 1 dimension.

Aspects of the disclosure provided herein comprise a method of training a predictive model to output a phenotypic characterization of one or more subjects' topographic dataset, comprising: (a) receiving one or more biological samples and phenotypic characterizations from a first set of subjects; (b) determining a first topographic dataset from the first set of subjects' one or more biological samples; (c) calculating a first set of features of the first topographic dataset; and (d) training a predictive model with the first set of features and the phenotypic characterizations of the first set of subjects, thereby generating a trained predictive model configured to output a phenotypic characterization of a second set of one or more subjects when the trained predictive model is inputted with a second set of features of a second topographic dataset. In some embodiments, the one or more biological samples comprise a tooth. In some embodiments, the first set of features of the topographic dataset comprise a number of peaks of the topographic dataset detected, the slope of one or more of the peaks, a two-dimensional gradient across the topographic data, or any combination thereof features of the topographic dataset. In some embodiments, the predictive model comprises a machine learning algorithm. In some embodiments, the machine learning algorithm comprises a support vector machine (SVM), naïve Bayes classification, random forest, neural network, deep neural network (DNN), recurrent neural network (RNN), deep RNN, a long short-term memory (LSTM) recurrent neural network (RNN), a gated recurrent unit (GRU), supervised learning algorithm, unsupervised machine learning algorithm, or any combination thereof. In some embodiments, the phenotypic characterization comprises the presence or lack thereof a disease or disorder, wherein the disease or disorder comprises autism spectrum disorder (ASD), attention deficit hyperactivity disorder (ADHD), amyotrophic lateral sclerosis (ALS), schizophrenia, irritable bowel disease (IBD), pediatric kidney disease, kidney transplant rejection, pediatric cancer, or any combination thereof.

Aspects of the disclosure provided herein comprise a method of training a predictive model to output a phenotypic characterization of one or more subjects' phenotype data, comprising: (a) imaging one or more biological samples of a first set of subjects, thereby generating a first topographic dataset; (b) calculating a first set of features of the first topographic dataset; and (c) training a predictive model with the first set of features and phenotypic characterizations of the first set of subjects, thereby generating a trained predictive model configured to output a phenotypic characterization of a second set of one or more subjects when the trained predictive model is inputted with a second set of feature of a second topographic dataset. In some embodiments, the first set and second set of subjects are the same or different. In some embodiments, the one or more biological sample comprise a tooth. In some embodiments, the first set or second set of features of the topographic dataset comprise a number of peaks of the topographic dataset detected, the slope of one or more of the peaks, a two-dimensional gradient across the topographic data, or any combination thereof features of the topographic dataset. In some embodiments, the first or second set of phenotypic characterizations comprises the presence or lack thereof a disease or disorder, wherein the disease or disorder comprises autism spectrum disorder (ASD), attention deficit hyperactivity disorder (ADHD), amyotrophic lateral sclerosis (ALS), schizophrenia, irritable bowel disease (IBD), pediatric kidney disease, kidney transplant rejection, pediatric cancer, or any combination thereof. In some embodiments, the predictive model comprises a machine learning algorithm. In some embodiments, the machine learning algorithm comprises a support vector machine (SVM), naïve Bayes classification, random forest, neural network, deep neural network (DNN), recurrent neural network (RNN), deep RNN, a long short-term memory (LSTM) recurrent neural network (RNN), a gated recurrent unit (GRU), supervised learning algorithm, unsupervised machine learning algorithm, or any combination thereof.

Aspects of the disclosure provided herein comprise a method of predicting a phenotypic classification of one or more subjects' topographic dataset, comprising: (a) imaging one or more biological samples of one or more subjects, thereby generating a topographic dataset; (b) calculating a set of features of the topographic dataset; and (c) predicting a phenotypic characterization as an output of a trained predictive model when the trained predictive model is provided the one or more subjects' set of features as an input. In some embodiments, the one or more biological sample comprise a tooth. In some embodiments, the set of features of the topographic dataset comprise a number of peaks of the topographic dataset detected, the slope of one or more of the peaks, a two-dimensional gradient across the topographic data, or any combination thereof features of the topographic dataset. In some embodiments, the phenotypic characterization comprises the presence or lack thereof a disease or disorder, wherein the disease or disorder comprises autism spectrum disorder (ASD), attention deficit hyperactivity disorder (ADHD), amyotrophic lateral sclerosis (ALS), schizophrenia, irritable bowel disease (IBD), pediatric kidney disease, kidney transplant rejection, pediatric cancer, or any combination thereof. In some embodiments, the predictive model comprises a machine learning algorithm. In some embodiments, the machine learning algorithm comprises a support vector machine (SVM), naïve Bayes classification, random forest, neural network, deep neural network (DNN), recurrent neural network (RNN), deep RNN, a long short-term memory (LSTM) recurrent neural network (RNN), a gated recurrent unit (GRU), supervised learning algorithm, unsupervised machine learning algorithm, or any combination thereof.

Aspects of the disclosure provided herein comprise a method of predicting a phenotypic characterization of one or more subjects' topographic dataset, comprising: (a) receiving one or more biological samples and phenotypic data of one or more subjects'; (b) determining a topographic dataset from the one or more subjects' one or more biological samples; (c) calculating a set of features of the first topographic dataset; and (d) predicting a phenotypic classification as an output of a trained predictive model when the trained predictive model is provided the one or more subjects' set of features as an input. In some embodiments, the one or more biological samples comprise a tooth. In some embodiments, the set of features of the topographic dataset comprise a number of peaks of the topographic dataset detected, the slope of one or more of the peaks, a two-dimensional gradient across the topographic data, or any combination thereof features of the topographic dataset. In some embodiments, the phenotypic characterization comprises the presence or lack thereof a disease or disorder, wherein the disease or disorder comprises autism spectrum disorder (ASD), attention deficit hyperactivity disorder (ADHD), amyotrophic lateral sclerosis (ALS), schizophrenia, irritable bowel disease (IBD), pediatric kidney disease, kidney transplant rejection, pediatric cancer, or any combination thereof. In some embodiments, the trained predictive model comprises a machine learning algorithm. In some embodiments, the machine learning algorithm comprises a support vector machine (SVM), naïve Bayes classification, random forest, neural network, deep neural network (DNN), recurrent neural network (RNN), deep RNN, a long short-term memory (LSTM) recurrent neural network (RNN), a gated recurrent unit (GRU), supervised learning algorithm, unsupervised machine learning algorithm, or any combination thereof.

Aspects of the disclosure provided herein comprise a method for outputting a feature importance score of one or more features of a subject correlated to phenotypic characterization, comprising: (a) receiving a biological sample of a subject; (b) contacting a surface of the biological sample with a compressible body optically coupled to at least one light source and at least one sensor, wherein the at least one light source generates a multi-dimensional dataset of a surface of the biological sample detected by the at least one sensor; (c) analyzing the multi-dimensional dataset, thereby determining one or more features of the multi-dimensional dataset of the surface of the biological sample; and (d) outputting a feature importance score of one or more features of the topographical dataset correlated to a phenotypic characterization. In some embodiments, the biological samples comprise a tooth. In some embodiments, the one or more of features of the multi-dimensional dataset comprise a number of peaks of the topographic dataset detected, the slope of one or more of the peaks, a two-dimensional gradient across the topographic data, or any combination thereof features of the topographic dataset. In some embodiments, the phenotypic characterization comprises the presence or lack thereof a disease or disorder, wherein the disease or disorder comprises autism spectrum disorder (ASD), attention deficit hyperactivity disorder (ADHD), amyotrophic lateral sclerosis (ALS), schizophrenia, irritable bowel disease (IBD), pediatric kidney disease, kidney transplant rejection, pediatric cancer, or any combination thereof. In some embodiments, the feature importance score is outputted by a trained predictive model, wherein the trained predictive model comprises a machine learning algorithm. In some embodiments, the machine learning algorithm comprises a support vector machine (SVM), naïve Bayes classification, random forest, neural network, deep neural network (DNN), recurrent neural network (RNN), deep RNN, a long short-term memory (LSTM) recurrent neural network (RNN), a gated recurrent unit (GRU), supervised learning algorithm, unsupervised machine learning algorithm, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example device setup to collect the topographic data of an ex-vivo biological sample surface, as described in some embodiments herein.

FIG. 2 shows a flow diagram for a method of collecting a biological surface's topographic data of an ex-vivo biological sample, as described in some embodiments herein.

FIGS. 3A-3B show an example dataset generated through an application of the device, methods, and systems to predict anthropomorphic and clinical phenotypic characteristics (height and/or weight) in one population of children (Study 1) from datasets of biological surface topographic data. Plots demonstrate device performance by illustrating the correlation between predicted height (FIG. 3A) and height measured in clinical assessment, and predicted weight (FIG. 3B) and weight measured at clinical assessment.

FIGS. 4A-4B show an example dataset generated through an application of the device, methods, and systems to predict anthropomorphic and clinical phenotypic characteristics (height and/or body mass index (BMI)) in one population of children (Study 2) from datasets of biological surface topographic data. Plots demonstrate device performance by illustrating the correlation between predicted height (FIG. 4A) and height measured in clinical assessment, and predicted BMI (FIG. 4B) and BMI measured in clinical assessment.

FIGS. 5A-5B show an example dataset generated through the application of the device, methods, and systems to predict intelligence quotient (IQ) (FIG. 5A) and child cognitive composite score (FIG. 5B) from datasets of biological surface topographic data. Plots demonstrate device performance by illustrating the correlation between predicted scores on these measures, based on analysis of surface topographic data, and scores measured through clinical assessment.

FIG. 6 illustrates a diagram of a system configured to process, execute, and/or implement the methods of the disclosure provided herein.

FIGS. 7A-7I show an example dataset generated through the application of the device, methods, and systems to predict the concentration of essential and non-essential elements in tooth samples from datasets of biological surface topographic data. Plots demonstrate device performance for multiple elements by illustrating the correlation between predicted elemental concentrations, based on analysis of surface topographic data, as compared to detecting the essential and non-essential elements using standard mass spectrometry methods, as described in some embodiments herein.

FIGS. 8A-8B illustrates use of the hand held device to measure the topography of an in-vivo biological surface, as described in some embodiments herein.

### DETAILED DESCRIPTION

The disclosure provided herein describes devices, systems, and methods related to measuring, capturing, and analyzing the topography of a biological surface. In some cases, the topography of the biological surface may be an in-vivo or an ex-vivo biological surface of a subject. The devices described herein may comprise hand held or bench-top devices. The disclosure provided herein may describe analytical methods applied to the measured topography of the biological surface. In some instances, the analytical methods may comprise training a predictive model with one or more features of the measured topography of the biological surface and a corresponding phenotype characteristic associated with the subject's biological surface measured. In some cases, the phenotype characteristic may comprise a subject's intelligence quotient (IQ), body mass index (BMI), height, weight, etc.

### Devices and Systems

The disclosure provided herein describes devices and systems that may be utilized to determine the topography of a biological surface. The device 100 may comprise at least one light source, at least one sensor, an optical assembly, and optionally a compressible body 106 optically coupled to the at least one light source, optical assembly, and/or the at least one sensor.

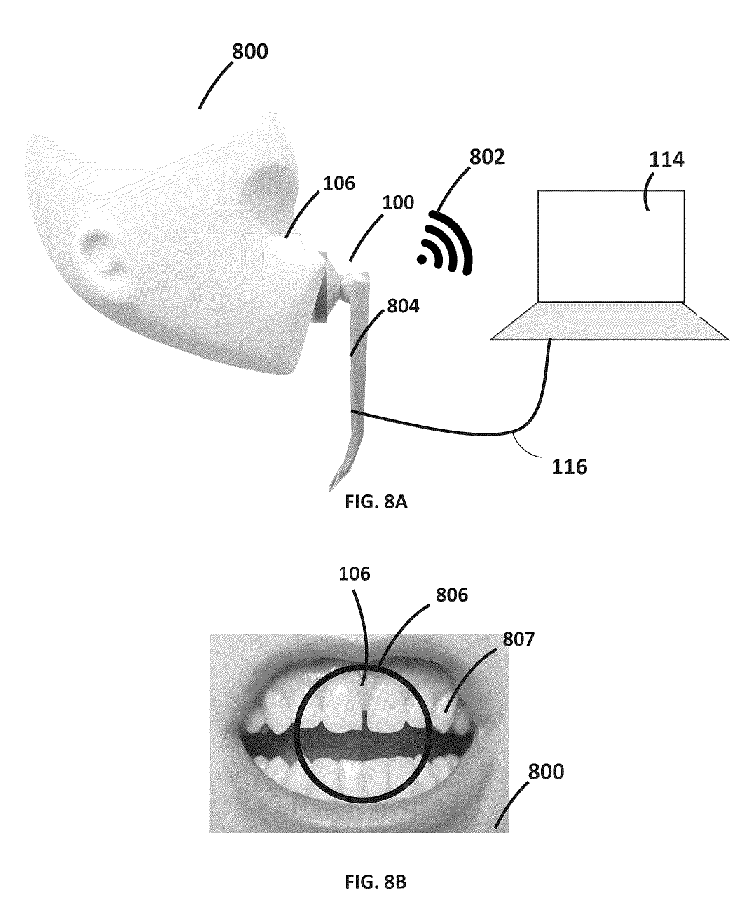

In some cases, the device may be a hand-held device 100, as seen in FIGS. 8A-8B, configured to be placed minimally or non-invasively over or in contact with a biological surface 806 (FIG. 8B) of a subject 800. In some cases, the biological surface 806 may be contacted by the device through an interface of a compressible body 106, described elsewhere herein. In some cases, the hand-held device may provide access to in-vivo biological surfaces within the oral cavity of a subject. In some cases, the biological surface may comprise a surface of one or more of a subject's teeth 807. The device 100 used in a hand-held configuration, as shown in FIG. 8A, may be in electrical communication via a wired 116 or wireless 802 communication with a processor 114. In some cases, the hand held device may comprise a magnetic, gyroscopic, accelerometer, or any combination thereof sensors to determine a three-dimensional position of the device in space. In some instances, the three-dimensional position in space of the device may be used to reconstruct data collected of a plurality of continuous and/or discontinuous biological surfaces.

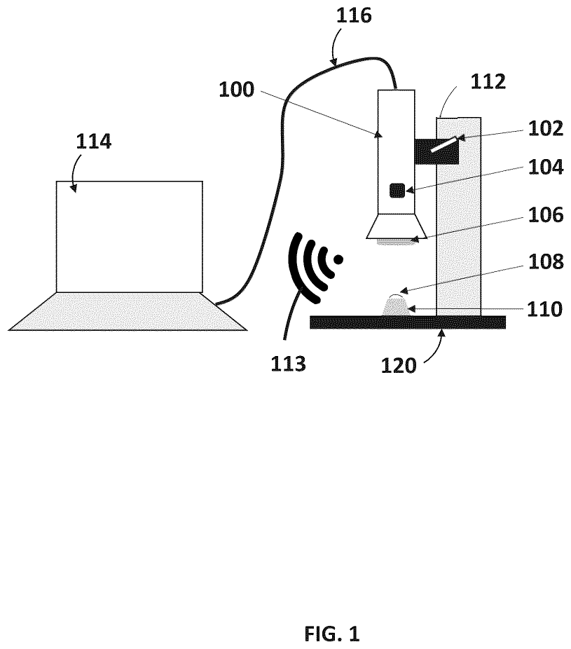

Alternatively the device may be a bench-top system, as seen in FIG. 1. In some instances, the device 100 may dock to a bench-top system (112, 120, 102) as shown in FIG. 1, to be used as a bench-top system. In some instances, the device 100 may be electrical communication via a wired 116 or wireless 113 communication platform with a processor 114. In some cases, the processor may comprise a personal desktop computer, server, cloud-based server, laptop computer, tablet computer, smart-phone, or any combination thereof. In some instances, the processor may initiate the collection of or analyze the biological surface topographic data collected by the device 100 of a biological sample 108. In some cases, the biological surface topographic data may comprise at multi-dimensional dataset. In some cases, the multi-dimensional data set comprises at least one dimension of data, at least two dimensions, or at least three dimensions of data. In some cases, the data may be converted to a one or more color and/or gray scale images.

In some instances, the device 100 may comprise an enclosure that houses electrical, optical, and/or mechanical components. In some cases, the enclosure may comprise a grip or ergonomic hand held feature 804 to allow for a user to hold and position the device over or in contact with a biological surface 806 of a subject such that the compress, as can be seen in FIGS. 8A-8B. In some cases, the device may comprise a battery, charging circuitry, a battery indicator, or any combination thereof configured to allow the device to be used in a hand-held manner.

In some cases, the at least one light source may comprise a coherent laser, incoherent laser, pulsed laser, confocal laser, light emitting diode (LED), organic light emitting diode (OLED), super-luminescent diode, or any combination thereof.

In some instances, the at least one light source emits light within a wavelength range of about 350 nm to about 1,000 nm. In some instances, the at least one light source emits light within a wavelength range of about 350 nm to about 400 nm, about 350 nm to about 440 nm, about 350 nm to about 480 nm, about 350 nm to about 520 nm, about 350 nm to about 560 nm, about 350 nm to about 600 nm, about 350 nm to about 640 nm, about 350 nm to about 670 nm, about 350 nm to about 600 nm, about 350 nm to about 800 nm, about 350 nm to about 1,000 nm, about 400 nm to about 440 nm, about 400 nm to about 480 nm, about 400 nm to about 520 nm, about 400 nm to about 560 nm, about 400 nm to about 600 nm, about 400 nm to about 640 nm, about 400 nm to about 670 nm, about 400 nm to about 600 nm, about 400 nm to about 800 nm, about 400 nm to about 1,000 nm, about 440 nm to about 480 nm, about 440 nm to about 520 nm, about 440 nm to about 560 nm, about 440 nm to about 600 nm, about 440 nm to about 640 nm, about 440 nm to about 670 nm, about 440 nm to about 600 nm, about 440 nm to about 800 nm, about 440 nm to about 1,000 nm, about 480 nm to about 520 nm, about 480 nm to about 560 nm, about 480 nm to about 600 nm, about 480 nm to about 640 nm, about 480 nm to about 670 nm, about 480 nm to about 600 nm, about 480 nm to about 800 nm, about 480 nm to about 1,000 nm, about 520 nm to about 560 nm, about 520 nm to about 600 nm, about 520 nm to about 640 nm, about 520 nm to about 670 nm, about 520 nm to about 600 nm, about 520 nm to about 800 nm, about 520 nm to about 1,000 nm, about 560 nm to about 600 nm, about 560 nm to about 640 nm, about 560 nm to about 670 nm, about 560 nm to about 600 nm, about 560 nm to about 800 nm, about 560 nm to about 1,000 nm, about 600 nm to about 640 nm, about 600 nm to about 670 nm, about 600 nm to about 600 nm, about 600 mm to about 800 nm, about 600 nm to about 1,000 nm, about 640 nm to about 670 nm, about 640 nm to about 600 nm, about 640 nm to about 800 nm, about 640 nm to about 1,000 nm, about 670 nm to about 600 nm, about 670 nm to about 800 nm, about 670 nm to about 1,000 nm, about 600 nm to about 800 nm, about 600 nm to about 1,000 nm, or about 800 nm to about 1,000 nm. In some instances, the at least one light source emits light within a wavelength range of about 350 nm, about 400 mm, about 440 nm, about 480 nm, about 520 nm, about 560 nm, about 600 nm, about 640 nm, about 670 nm, about 600 mm, about 800 nm, or about 1,000 nm. In some instances, the at least one light source emits light within a wavelength range of at least about 350 nm, about 400 nm, about 440 nm, about 480 mm, about 520 mm, about 560 nm, about 600 nm, about 640 mm, about 670 nm, about 600 nm, or about 800 nm. In some instances, the at least one light source emits light within a wavelength range of at most about 400 mm, about 440 nm, about 480 nm, about 520 nm, about 560 nm, about 600 nm, about 640 nm, about 670 mm, about 600 nm, about 800 mm, or about 1,000 nm.

In some cases, the at least one sensor comprises an elastomeric gel membrane, camera, photomultiplier tube, charged coupled device (CCD), complementary metal-oxide-semiconductor (CMOS), or any combination thereof sensors. In some cases, the at least one sensor may be configured to capture a three-dimensional spatial dataset of the biological surface topographic data. In some cases, the sensor may be configured to capture at least a one, two, or three-dimensional spatial dataset of the biological surface topographic data.

In some cases, the at least one sensor may be configured to produce a dataset with a spatial resolution of about 0.01 μm to about 2 μm. In some cases, the at least one sensor may be configured to produce a dataset with a spatial resolution of about 0.01 μm to about 0.03 μm, about 0.01 μm to about 0.05 μm, about 0.01 μm to about 0.08 μm, about 0.01 μm to about 0.1 μm, about 0.01 μm to about 0.5 μm, about 0.01 μm to about 0.8 μm, about 0.01 μm to about 1 μm, about 0.01 μm to about 1.2 μm, about 0.01 μm to about 1.5 μm, about 0.01 μm to about 1.7 μm, about 0.01 μm to about 2 μm, about 0.03 μm to about 0.05 μm, about 0.03 μm to about 0.08 μm, about 0.03 μm to about 0.1 μm, about 0.03 μm to about 0.5 μm, about 0.03 μm to about 0.8 μm, about 0.03 μm to about 1 μm, about 0.03 μm to about 1.2 μm, about 0.03 μm to about 1.5 μm, about 0.03 μm to about 1.7 μm, about 0.03 μm to about 2 μm, about 0.05 μm to about 0.08 μm, about 0.05 μm to about 0.1 μm, about 0.05 μm to about 0.5 μm, about 0.05 μm to about 0.8 μm, about 0.05 μm to about 1 μm, about 0.05 μm to about 1.2 μm, about 0.05 μm to about 1.5 μm, about 0.05 μm to about 1.7 μm, about 0.05 μm to about 2 μm, about 0.08 μm to about 0.1 μm, about 0.08 μm to about 0.5 μm, about 0.08 μm to about 0.8 μm, about 0.08 μm to about 1 μm, about 0.08 μm to about 1.2 μm, about 0.08 μm to about 1.5 μm, about 0.08 μm to about 1.7 μm, about 0.08 μm to about 2 μm, about 0.1 μm to about 0.5 μm, about 0.1 μm to about 0.8 μm, about 0.1 μm to about 1 μm, about 0.1 μm to about 1.2 μm, about 0.1 μm to about 1.5 μm, about 0.1 μm to about 1.7 μm, about 0.1 μm to about 2 μm, about 0.5 μm to about 0.8 μm, about 0.5 μm to about 1 μm, about 0.5 μm to about 1.2 μm, about 0.5 μm to about 1.5 μm, about 0.5 μm to about 1.7 μm, about 0.5 μm to about 2 μm, about 0.8 μm to about 1 μm, about 0.8 μm to about 1.2 μm, about 0.8 μm to about 1.5 μm, about 0.8 μm to about 1.7 μm, about 0.8 μm to about 2 μm, about 1 μm to about 1.2 μm, about 1 μm to about 1.5 μm, about 1 μm to about 1.7 μm, about 1 μm to about 2 μm, about 1.2 μm to about 1.5 μm, about 1.2 μm to about 1.7 μm, about 1.2 μm to about 2 μm, about 1.5 μm to about 1.7 μm, about 1.5 μm to about 2 μm, or about 1.7 μm to about 2 μm. In some cases, the at least one sensor may be configured to produce a dataset with a spatial resolution of about 0.01 μm, about 0.03 μm, about 0.05 μm, about 0.08 μm, about 0.1 μm, about 0.5 μm, about 0.8 μm, about 1 μm, about 1.2 μm, about 1.5 μm, about 1.7 μm, or about 2 μm. In some cases, the at least one sensor may be configured to produce a dataset with a spatial resolution of at least about 0.01 μm, about 0.03 μm, about 0.05 μm, about 0.08 μm, about 0.1 μm, about 0.5 μm, about 0.8 μm, about 1 μm, about 1.2 μm, about 1.5 μm, or about 1.7 μm. In some cases, the at least one sensor may be configured to produce a dataset with a spatial resolution of at most about 0.03 μm, about 0.05 μm, about 0.08 μm, about 0.1 μm, about 0.5 μm, about 0.8 μm, about 1 μm, about 1.2 μm, about 1.5 μm, about 1.7 μm, or about 2 μm.

In some cases, the compressible body 106 may comprise a compressible gel cartridge comprising a deformable elastomer gel substrate and membrane configured to amplify the biological sample 108 surface's topography when brought into contact with the biological sample 108. In some instances, the deformable plastic may comprise materials of thermoplastic elastomer (TPE) or silicone. In some cases, the device may operate without the use of a compressible body 106 by removing the compressible cartridge 106 from the optical path of the device 100. In some cases, the device may comprise a switch or actionable button 104 configured to initiate, pause, or end collection of the biological surface topographic data. In some cases, the switch or actionable button 104 may comprise a physical push button, force sensor, capacitive touch button, or any combination thereof. In some instances, a user by pressing or activating the switch or actionable button 104 for an extended period of time may trigger different functionality of the device and/or system (e.g., particular light source illumination patterns or averaging of data collected).

In some cases, the device may comprise an optical assembly comprising one or more lenses or lens elements. In some cases, the one or more lenses may comprise a concave lens, convex lens, bi-concave, bi-convex, planoconcave, planoconvex, spherical or any combination thereof lenses. In some instances, the one or more lenses may be configured to direct, expand, collimate, focus, or any combination thereof light ray manipulation of the at least one light source light emission or the reflected light off of the biological sample 108 collected by the at least one sensor. In cases, the one or more lenses may comprise an anti-reflective coating configured to reflect and/or transmit light of specific bandwidths of light. In some cases, the optical assembly may further comprise a dichroic filter, hot mirror, cold mirror, or any combination thereof mirrors.

### Systems

The disclosure provided herein describes systems configured to determine a topography of a biological surface. In some instances, the systems may comprise a hand held system configured to image an in-vivo biological surface topography. Alternatively, the systems may comprise a bench-top system configured to image ex-vivo biological surface topography. In some cases, the system may comprise: (a) a fixture mechanically configured to constrain a three-dimensional position of a biological sample; (b) a compressible body optically coupled to at least one light source and at least one sensor, where the at least one light source generates a multi-dimensional dataset of the surface of the biological sample detected by the at least one sensor; and (c) a processor electrically coupled to the at least one light source and the least one sensor, where the processor comprises a set of programmed instructions stored on a non-transitory storage medium configured to cause the processor to determine a topography of the surface of the biological sample from the multi-dimensional dataset. In some cases, the biological sample may be a tooth. In some cases, the compressible body may comprise a gel cartridge. In some cases, the compressible body is housed in a locking mechanical member configured to fix the three-dimensional position of the compressible body.

In some instances, the fixture 110 may comprise putty, a mechanical mount, adhesive compound, or any combination thereof. In some instances, the device 100 may be mechanically coupled to a mounting feature 102 configured to releasably lock the position of the device with respect to the biological sample 108. In some instances, the spatial position of the compressible body of the device may be releasably lockable by a locking feature of the mounting feature 102. In some cases, the locking feature may comprise a quick release or latch based locking featuring. In some instances, the locking feature may fix the three-dimensional position of the compressible body with respect to the biological sample. In some instances, the mounting feature may be mechanically coupled to a rigid guide member 112, allowing for the mounting feature to move in a constrained dimension along the rigid guide member 112 towards or away from the mounted biological sample 108. In some cases, the rigid guide member 112 may comprise a track, rail, or post 112. In some instances, the rigid guide member may be mechanically coupled to a base 120 configured to reduce vibrations when collecting data of the biological surface topography using the device mounted in a bench-top system as shown in FIG. 1.

In some cases, the at least one light source may comprise at least one coherent laser, incoherent laser, pulsed laser, light emitting diode, super-luminescent diode, or any combination thereof. In some cases, the at least one light source and the at least one sensor are in electrical communication with a button, switch, trigger (104), or any combination thereof configured to initiate illumination of the biological sample and detection of the multi-dimensional dataset of the surface of the biological sample. In some cases, the multi-dimensional dataset comprises at least 1 dimension.

### Computer Systems

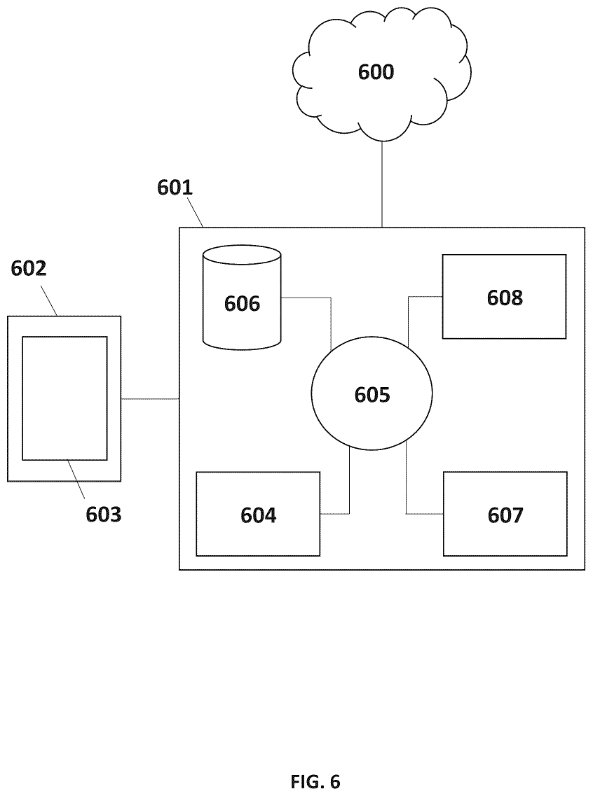

FIG. 6 shows a computer system 601 suitable for implementing and/or training models and/or predictive models described herein. The computer system 601 may process various aspects of data and/or information of the present disclosure, such as, for example, subjects' biological sample topography raw data, topography generated images, processed topography data parameters or features, corresponding subject phenotypic characterizations, clinical meta data, or any combination thereof. The computer system 601 may be an electronic device. The electronic device may be a mobile electronic device.

The computer system 601 may comprise a central processing unit (CPU, also “processor” and “computer processor” herein) 605, which may be a single core or multi core processor, or a plurality of processor for parallel processing. The computer system 601 may further comprise memory or memory locations 604 (e.g., random-access memory, read-only memory, flash memory), electronic storage unit 606 (e.g., hard disk), communications interface 608 (e.g., network adapter) for communicating with one or more other devices, and peripheral devices 607, such as cache, other memory, data storage and/or electronic display adapters. The memory 604, storage unit 606, interface 608, and peripheral devices 607 are in communication with the CPU 605 through a communication bus (solid lines), such as a motherboard. The storage unit 606 may be a data storage unit (or a data repository) for storing data. The computer system 601 may be operatively coupled to a computer network (“network”) 600 with the aid of the communication interface 608. The network 600 may be the Internet, an internet and/or extranet, or an intranet and/or extranet that is in communication with the Internet. The network 600 may, in some case, be a telecommunication and/or data network. The network 600 may include one or more computer servers, which may enable distributed computing, such as cloud computing. The network 600, in some cases with the aid of the computer system 601, may implement a peer-to-peer network, which may enable devices coupled to the computer system 601 to behave as a client or a server.

The CPU 605 may execute a sequence of machine-readable instructions, which may be embodied in a program or software. The instructions may be directed to the CPU 605, which may subsequently program or otherwise configured the CPU 605 to implement methods of the present disclosure. Examples of operations performed by the CPU 605 may include fetch, decode, execute, and writeback.

The CPU 605 may be part of a circuit, such as an integrated circuit. One or more other components of the system 601 may be included in the circuit. In some cases, the circuit is an application specific integrated circuit (ASIC).

The storage unit 606 may store files, such as drivers, libraries and saved programs. The storage unit 606 may store subjects' biological sample topography raw data, topography generated images, processed topography data parameters or features, corresponding phenotypic characterizations, clinical meta data, or any combination thereof. The computer system 601, in some cases may include one or more additional data storage units that are external to the computer system 601, such as located on a remote server that is in communication with the computer system 601 through an intranet or the internet.

Methods as described herein may be implemented by way of machine (e.g., computer processor) executable code stored on an electronic storage location of the computer device 601, such as, for example, on the memory 604 or electronic storage unit 606. The machine executable or machine-readable code may be provided in the form of software. During use, the code may be executed by the processor 605. In some instances, the code may be retrieved from the storage unit 606 and stored on the memory 604 for ready access by the processor 605. In some instances, the electronic storage unit 606 may be precluded, and machine-executable instructions are stored on memory 604.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 601, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 602 that comprises a user interface (UI) 603 for viewing a phenotypic characterization prediction of a subject based on their biological sample topographic data. Examples of UI's include, without limitation, a graphical user interface (GUI) and web-based user interface.

Methods and systems of the present disclosure can be implemented by way of one or more algorithms and/or predictive models with instructions provided with one or more processors as disclosed herein. An algorithm and/or predictive model can be implemented by way of software upon execution by the central processing unit 905. In some cases, the predictive model may comprise a machine learning predictive model. In some cases, the machine learning predictive model may comprise one or more statistical, machine learning, or artificial intelligence algorithms. Examples of utilized algorithms may include a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), or a gated recurrent unit (GRU), supervised learning unsupervised machine learning, statistical, deep-learning algorithm for classification and regression. The machine learning predictive model may likewise involve the estimation of ensemble models, comprised of multiple predictive models, and utilize techniques such as gradient boosting, for example in the construction of gradient-boosting decision trees. The machine learning predictive model may be trained using one or more training datasets corresponding to a subject's data. In some embodiments, the one or more training datasets may comprise exposome biochemical signatures, dynamic exposome biochemical signatures, clinical metadata, clinical trial information, exposome biochemical signature information of pharmaceutical and nutraceutical treatments, or any combination thereof.

### Methods

The disclosure provided herein describes methods of collecting or determining biological surface topographic data from a biological sample of a subject. In some instances the methods of the disclosure provided herein may comprise methods of generating, calculating, determining features or characteristics of a subject's biological surface topographic data that may be used in combination with a subject's phenotypic characterization to train a predictive model. In some cases, the predictive model may comprise a machine learning algorithm, described elsewhere herein. In some instances, a subject's phenotypic characterization may comprise the presence or lack thereof disease state (e.g., autism, ADHD, cancer, etc.), physiologic parameter (e.g., weight, height, body mass index), or any combination thereof.

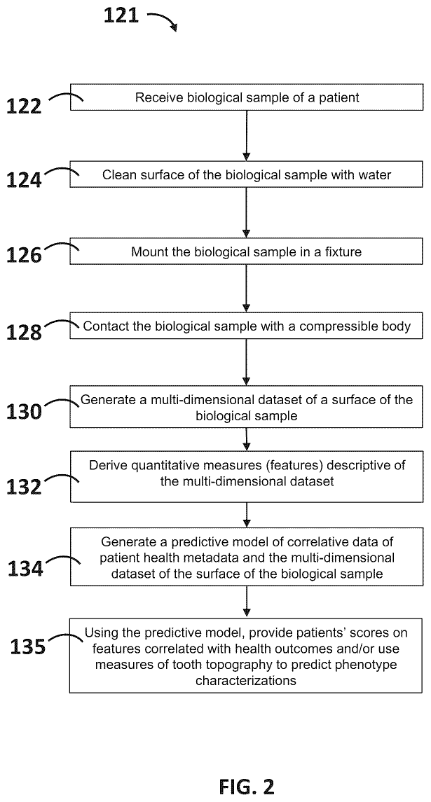

In some cases, the disclosure provided herein describes a method for determining a topography of a biological sample 121, as seen in FIG. 2. In some cases, the method may comprise the steps of (a) receiving a biological sample of a subject 122; (b) mounting the biological sample into a fixture 126; (c) contacting the biological sample with a compressible body 128 optically coupled to at least one light source and at least one sensor; (d) generating a multi-dimensional dataset of the surface of the biological sample 130; (e) derive quantitative measures (i.e., features) descriptive of the multi-dimensional dataset 132; (f) generate a predictive model based on correlative data of subject health metadata and the quantitative measures of the multi-dimensional dataset 134; and (g) using the predictive model, provide subject's scores for features correlated with health outcomes and/or measures of tooth topography to predict phenotypic characterizations 135. In some cases, step (a)-(c) may be replaced by imaging a biological sample, thereby generating a multi-dimensional dataset of the biological sample's surface. In some cases, the method may further comprise cleaning 124 the surface of the biological sample to (b) mounting the biological sample into a fixture. In some instances, the cleaning the surface of the biological sample may remove dirt, debris, or other particles that may negatively influence the measured topography of the biological sample surface. In some instances, the cleaning of the surface of the biological sample may be cleaned using a damp lint free cloth or fabric that will not leave a residue upon cleaning. In some cases, deionized water may be used with the Kimwipe to clean to the surface of the biological sample. In some instances, the biological sample may comprise an ex-vivo or in-vivo biological sample (e.g., an extracted tooth of a subject compared to a tooth of a subject in the subject's mouth). In some cases, the method may further comprise calibrating the compressible body on a standard glass, ball grid array (BGA), groove targets, validation plate, or any combination thereof prior to contacting the biological sample with the compressible body. In some instances, the spatial position of the compressible body may be releasably lockable.

In some cases, the fixture 110 may comprise a putty, mechanical mount, adhesive compound, or any combination thereof. In some instances, the fixture may limit or constrain the motion of the biological sample when the device, described elsewhere herein, is used to determine a topography dataset of a surface of the biological sample. In some instances, the at least one light source and the at least one sensor may be electrically in communication with a button, switch, trigger, or any combination therefore configured to initiate illumination of the biological sample, pause illumination of the biological sample, stop illumination of the biological sample, initiate collection of the topography dataset by the at least one sensor, pause collection of the topography dataset by the at least one sensor, stop collection of the topography dataset by the at least one sensor, or any combination thereof actions. In some cases, the multi-dimensional dataset comprises at least 1 dimension. In some cases, the method may comprise the step of generating a trained predictive model by training a model with the topography dataset and corresponding subject health metadata.

In some cases, the disclosure provided herein describes a method for outputting a feature importance score of one or more features correlated to a phenotypic characterization of a subject. In some cases, the method may comprise the steps of (a) receiving a biological sample of a subject; (b) mounting the biological sample into a fixture; (c) contacting a surface of the biological sample with a compressible body optically coupled to at least one light source and at least one sensor, wherein the at least one light source generates a multi-dimensional dataset of the surface of the biological sample detected by the at least one sensor; (d) analyzing the multi-dimensional dataset thereby determining one or more features of the multi-dimensional dataset of the surface of the biological sample; and (e) outputting a feature importance score of one or more features of the topography dataset. In some cases, step (a)-(c) may be replaced by imaging one or more in-vivo biological samples of one or more subjects, thereby generating a multi-dimensional dataset of the one or more in-vivo biological samples' surfaces. In some cases, the method may further comprise cleaning the surface of the biological sample to (b) mounting the biological sample into a fixture. In some instances, the cleaning the surface of the biological sample may remove dirt, debris, or other particles that may negatively influence the measured topography of the biological sample surface. In some instances, the cleaning of the surface of the biological sample may be cleaned using a damp lint free cloth or fabric that will not leave a residue upon cleaning. In some cases, deionized water may be used with the Kimwipe to clean to the surface of the biological sample. In some instances, the biological sample may comprise an ex-vivo or in-vivo biological sample (e.g., an extracted tooth of a subject compared to a tooth of a subject in the subject's mouth). In some cases, the method may further comprise calibrating the compressible body on a standard glass, ball grid array (BGA), groove targets, validation plate, or any combination thereof prior to contacting the biological sample with the compressible body. In some instances, the spatial position of the compressible body may be releasably lockable.

In some cases, the one or more features of the topographic dataset may be correlated or associated to one or more exposomic signatures. In some instances, the one or more exposomic signatures may comprise metal ion concentration. In some cases, the metal ions may comprise ions of chemical elements, including zinc (Zn), lead (Pb), copper (Cu), arsenic (As), manganese (Mn), cadmium (Cd), magnesium (Mg), calcium (Ca), and chromium (Cr). In some instances, the temporal metal ion concentration are determined from a subject's biological sample using standard mass spectrometry methods.

In some cases, the fixture may comprise a putty, mechanical mount, adhesive compound, or any combination thereof. In some instances, the fixture may limit or constrain the motion of the biological sample when the device, described elsewhere herein, is used to determine a topography dataset of a surface of the biological sample. In some instances, the at least one light source and the at least one sensor may be electrically in communication with a button, switch, trigger, or any combination therefore configured to initiate illumination of the biological sample, pause illumination of the biological sample, stop illumination of the biological sample, initiate collection of the topography dataset by the at least one sensor, pause collection of the topography dataset by the at least one sensor, stop collection of the topography dataset by the at least one sensor, or any combination thereof actions. In some cases, the multi-dimensional dataset comprises at least 1 dimension. In some cases, the method may comprise the step of generating a trained predictive model by training a model with the topography dataset and corresponding subject health metadata.

In some cases, the disclosure provided herein describes a method of training a predictive model to output a phenotypic characterization of one or more subjects' phenotype characteristics. In some cases, the disclosure method may comprise the steps of: (a) receiving one or more ex-vivo biological samples and phenotypic characterization data from a first set of subjects; (b) determining a first topographic dataset of the first set of subjects' one or more biological samples' surfaces; (c) calculating a first set of features of the first topographic dataset; and (d) training a predictive model with the first set of features and the phenotypic data of the first set of subjects, thereby generating a trained predictive model configured to output a phenotypic characterization of a second set of one or more subjects when the trained predictive model is inputted with a second set of features of a second set of topographic data. In some cases, step (a) and (b) may be replaced by imaging one or more in-vivo biological samples of one or more subjects. In some instances, the first set and second set of subjects are same or different.

In some instances, the first or second set of features may comprise the number of peaks detected in a two-dimensional surface profile, peaks detected in a one dimensional surface profile, the slope of one or more peaks, a two-dimensional gradient across the topographic data, or any combination thereof features of the topographic data. In some instances, the first or second set of features may comprise measurements derived from application of recurrence quantification analysis to the surface topography data. In some instances, the first or second set of features may comprise measurements derived from application of recurrence quantification analysis to measures derived from the surface topography data; in particular, the intervals between peaks. In some instances, the first or second set of features may involve the derivation of measures of surface topography data involving aspects of information theory, particularly Shannon entropy in the surface topography waveform, or Shannon entropy in the intervals between peaks. In some cases, the first or second features may comprise a linear profile of topographic data. In some instances, the profile may be drawn on a user interface, described elsewhere herein, by a user or operator. In some instances, the linear profile may be taken from an anatomically co-registered region of the topographic dataset. In some cases, the anatomically co-registered region may comprise a linear segment between the incisal edge to the cervical end of the crown of the ex-vivo or in-vivo tooth. In some cases, the first or second set of features may comprise a roughness, average height, or any combination thereof parameter of the linear profile.

In some cases, the phenotypic characterizations of the first set of subjects may comprise the presence or lack thereof disease state (e.g., autism, ADHD, cancer, etc.), physiologic parameter (e.g., weight, height, body mass index), aspects of psychological development including intelligence quotient and/or cognitive measures, or any combination thereof. In some cases, determining a first topographic dataset from the first set of subjects' one or more biological samples may be accomplished by a bench-top system, described elsewhere herein.

In some cases, the disclosure provided herein describes a methods of predicting a phenotypic characterization of one or more subjects' topographic datasets. In some instances, the method may comprise the steps of: (a) receiving one or more biological samples and phenotypic data from one or more subjects; (b) determining topographic data from the one or more subjects' one or more biological samples; (c) calculating a set of features of the topographic data; and (d) predicting a phenotypic characterization as an output of a trained predictive model when the trained predictive model is provided as an input the one or more subjects' set of features as an input. In some cases, step (a) and (b) may be replaced by imaging one or more in-vivo biological samples of one or more subjects.

In some instances, the set of features may comprise the number of peaks detected, the slope of one or more peaks, a two-dimensional gradient across the topographic data, or any combination thereof features of the topographic data. In some instances, the first or second set of features may comprise measurements derived from application of recurrence quantification analysis (RQA) to the surface topography data.

RQA may include construction of recurrence plots that visualize and analyze features derived from the topographic surface data. Such recurrence plots may illustrate phasic processes in spatial measurements. From the spatial topography measured from a surface of a biological sample e.g., a tooth sample, additional dimensions may be computationally derived to embed the spatial measurement in a higher dimensional space referred to as a phase portrait, where x refers to the dimensions of the topographic dataset, and dimensions (x+τ) and (x+2τ) may be derived from original spatial dimensions offset by an interval τ. Subsequent analyses may then be undertaken on the embedded phase portrait to construct recurrence plots and recurrence quantification analysis. A recurrence quantification plot may be derived from the phase portrait through the application of a threshold function to each point in the phase portrait; on the corresponding recurrence plot, consisting of a square binary matrix, typically represented as white or black space, a given point is assigned a value of 1 at each spatial interval where another point in the phase-portrait shares the spatial limits of the assigned threshold boundary. The RQA methods may be applied to the recurrence plot to examine the interval between states in a given system, with a black point reflecting the spatial interval when a system revisits the same state. Periodic processes, where a system successively reiterates a given pattern of states, may manifest in a recurrence plot as diagonal black lines, whereas periods of stability may manifest as square structures, spurious repetitions as black dots, and, unique events as white space.

In some embodiments, the recurrence plots may be constructed for one spatial dimension or a combination of two or more dimensions of the topographic data (e.g., in order to visualize an interactive periodic pattern of two or more dimensions of the topographic dataset) this can be referred to as cross-recurrence quantification analysis, or joint-recurrence quantification analysis).

In some embodiments, the data analysis may include analyzing the recurrence plots to obtain a set of features associated with the recurrence plots. The features, which interchangeably can be termed “rhythmicity features,” or “dynamic features,” provide a quantitative measure describing the periodicity, predictability, and transitivity present in one or more dimensions of the topographic dataset. The features are selected from a set including recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, and/or any combination thereof.

In some instances, the first or second set of features may comprise measurements derived from application of recurrence quantification analysis (RQA) to measures derived from the surface topography data; in particular, the intervals between peaks. In some instances, the first or second set of features may involve the derivation of measures of surface topography data involving aspects of information theory, particularly Shannon entropy in the surface topography waveform, or Shannon entropy in the intervals between peaks. In some cases, the set of features may comprise a linear profile of topographic data. In some instances, the profile may be drawn on a user interface, described elsewhere herein, by a user or operator. In some instances, the linear profile may be taken from an anatomically co-registered region of the topographic dataset. In some cases, the anatomically co-registered region may comprise a linear segment between the incisal edge to the cervical end of the crown of the ex-vivo or in-vivo tooth. In some cases, the set of features may comprise a roughness, average height, or any combination thereof parameter of the linear profile.

In some instances, the application of recurrence quantification analysis to the surface topography data, or to derived features of the surface topography data such as peaks detected, the intervals between peaks, or the measured roughness of the surface topography, may yield measures including signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some instances, the derivation of features from the surface topography data, or from derived features of the surface topography data such as peaks detected, the intervals between peaks, may include the estimation of Lyapunov exponents, entropy estimation, cross convergent mapping, nonlinear modeling and parameter estimation, changepoint estimation, frequency-domain representation of the surface topography data or features derived from surface topography data, power-spectral domain representation of the surface topography data, features derived from surface topography data, or any combination thereof.

In some instances, recurrence matrices derived from analysis of the surface topography data, or to derived features of the surface topography data such as peaks detected, the intervals between peaks, or the measured roughness of the surface topography, may extend to network based models, wherein features descriptive of network connectivity, efficiency, feature importance, pathway importance, and related graph-theory based metrics may be derived.

Methods and features of RQA are described, for example, by Webber et al. in “Simpler Methods Do It Better: Success of Recurrence Quantification Analysis as a General Purpose Data Analysis Tool,” Physics Letters A 373, 3753-3756 (2009) and by Marwan et al. in “Recurrence Plots for the Analysis of Complex Systems,” Physics Reports 438, 237-239 (2007), the contents of each of which are herein incorporated by reference in their entirety. In some embodiments, the spatial dimensions of the topographic data are analyzed by using other analytical methods, such as Fourier Transformations, Wavelet Analysis, and Cosinor analysis. Such techniques can be applied to derive similar metrics, including spectral analysis of frequency components and their associated power. These metrics and associated derivative measures may be used in place of the features derived from RQA to analyze the one or more spatial dimensions of the topographic data obtained from a surface of a biological samples for purposes of predictive classification.

In some instances, the machine learning predictive model which utilizes features derived from the analysis of surface topography data may comprise one or more statistical, machine learning, or artificial intelligence algorithms. Examples of utilized algorithms may include gradient boosting ensemble learners, a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), or a gated recurrent unit (GRU), or other supervised learning algorithm or unsupervised machine learning, statistical, or deep-learning algorithm for classification and regression. The machine learning classifier may likewise involve the estimation of ensemble models, comprised of multiple predictive models, and utilize techniques such as gradient boosting, for example in the construction of gradient-boosting decision trees. The machine learning classifier may be trained using one or more training datasets corresponding to a subject's data. In some embodiments, the one or more training datasets may comprise exposome biochemical signatures, dynamic exposome biochemical signatures, clinical metadata, clinical trial information, exposome biochemical signature information of pharmaceutical and nutraceutical treatments, or any combination thereof.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks. Massachusetts Institute of Technology, each of which is hereby incorporated by reference.

SVMs are described in Cristianini and Shawe-Taylor, 2000, “An Introduction to Support Vector Machines,” Cambridge University Press, Cambridge: Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees are described generally by Duda, 2001. Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART. ID3, and C4.5 are described in Duda, 2001. Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag. New York, Chapter 9, which is hereby incorporated by reference in its entirety. Random Forests are described in Breiman, 1999, “Random Forests-Random Features,” Technical Report 567, Statistics Department, U. C. Berkeley, September 1999, which is hereby incorporated by reference in its entirety.

Clustering (e.g., unsupervised clustering model algorithms and supervised clustering model algorithms) is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y., and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5, which is hereby incorporated by reference in its entirety. In some embodiments, ensemble modeling techniques are used, for example, toward the classification algorithms described herein; these ensemble modeling techniques are described in the implementation of classification models herein, are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis may be performed by a device executing one or more programs (e.g., one or more programs stored in the Non-Persistent Memory (i.e., RAM or ROM) 604 or in the storage unit 606 (i.e., hard-disk) as seen in FIG. 6 including instructions to perform the data analysis. In some embodiments, the data analysis is performed by a system comprising at least one processor (e.g., the processing core 605) and memory (e.g., one or more programs stored in the Non-Persistent Memory 604 or in the storage unit 606) comprising instructions to perform the data analysis

In some cases, the phenotypic characterizations of the one or more subjects may comprise the presence or lack thereof a disease state (e.g., autism. ADHD, cancer, etc.), physiologic parameters (e.g., weight, height, body mass index), aspects of psychological development including intelligence quotient and/or cognitive measures, or any combination thereof. In some cases, determining the topographic dataset from the one or more subjects' one or more biological samples may be accomplished by a bench-top and/or handheld system, described elsewhere herein.

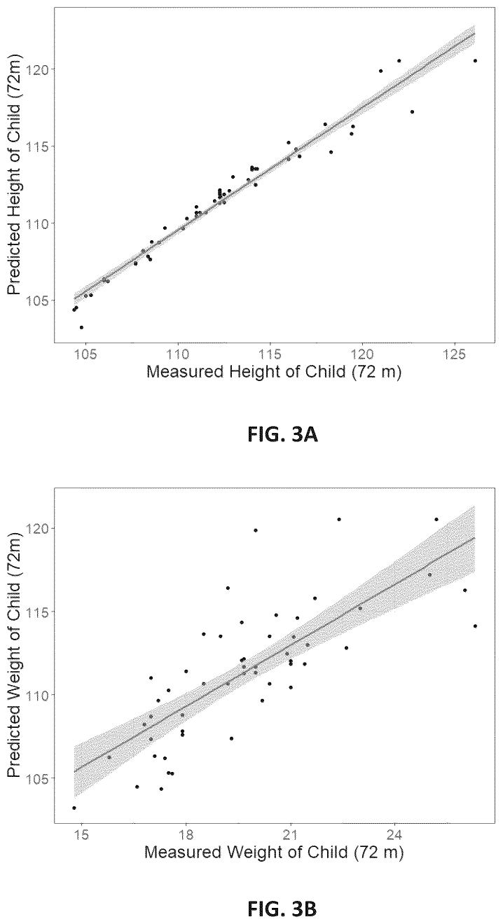

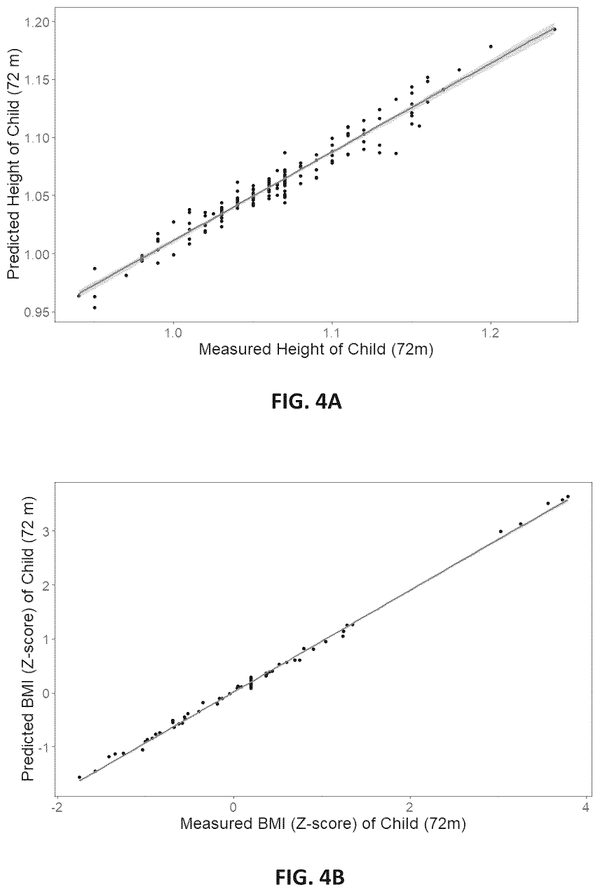

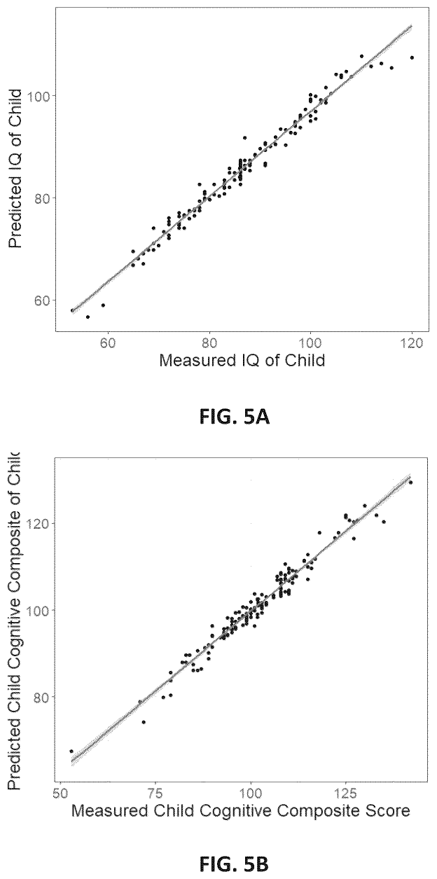

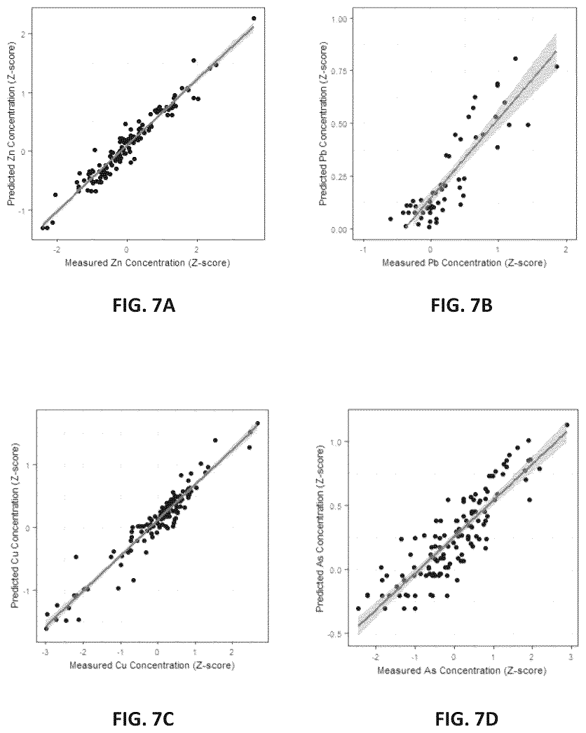

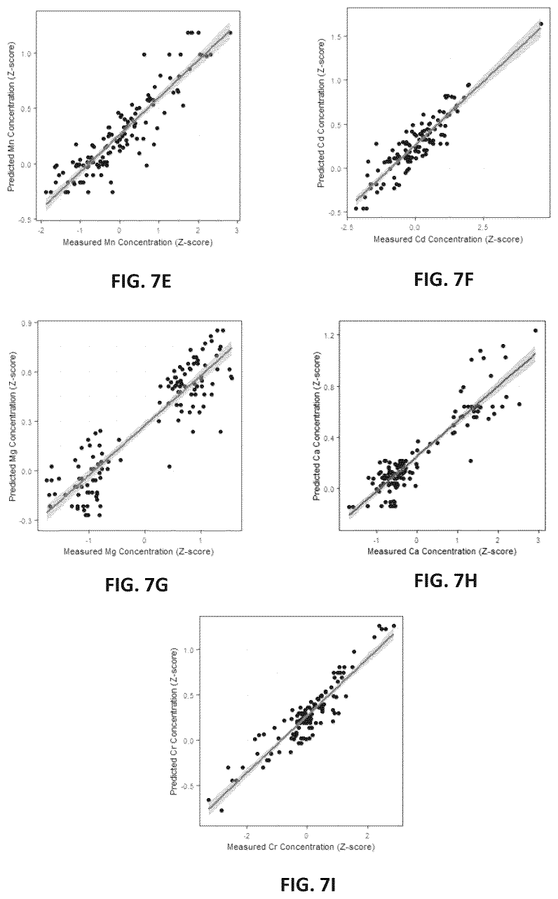

In some cases, the accuracy of one or more feature of the set of features calculated, described elsewhere herein, in predicting a phenotypic characteristic may be analyzed, as can be seen in experimental data shown in FIGS. 3A-3B, FIGS. 4A-4B, and FIGS. 5A-5B. In some cases, the one or more features of the set of features may predict the phenotypic characterization of intelligence quotient (IQ), as seen in FIG. 5A. Alternatively or in addition to the one or more features of the set of features may predict the phenotypic characterization of presence or lack thereof disease state e.g., autism, ADHD, cancer, physiologic parameter e.g., weight (FIG. 3B), height (FIG. 3A and FIG. 4A), body mass index (FIG. 4B), cognitive composite scores (FIG. 5B), or any combination thereof. In some instances, the device may utilize one or more features of the set of features to predict concentrations of chemical biomarkers, such as essential or non-essential elements, described elsewhere herein, that may be concentrated in a subject's biological tissue or tissue sample e.g., dentate tissue, as shown in FIGS. 7A-7I.

Although the above steps show each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations, and the program may comprise program instructions stored on a computer readable memory or programmed steps of the logic circuitry such as the programmable array logic or the field programmable gate array, for example.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying.” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1: Tooth Topographic Data as a Predictor for Measured Height and Weight of a Child

In a sample set of 52 subjects collected in a population-based cohort study, ex-vivo teeth were analyzed with the bench-top system, described elsewhere herein, with TPE gel cartridges. The ex-vivo teeth samples were obtained from one to five years from the moment of shedding. Each tooth was then scanned with the bench-top system, described elsewhere herein, to generate a 3-D topographic dataset of the top surface of the tooth. The 3-D topographic datasets were then analyzed, as described elsewhere herein, to generate one or more features of the 3-D topographic dataset. One of such features calculated from the 3-D topographic dataset included a linear profile of the 3-D topographic dataset measured from a tooth's incisal edge to the cervical end of the crown on the labial/buccal side of the crown. Other features descriptive of tooth topography were derived as described and used in the construction of model predictive of anthropometric features at 6 years of age, including height and weight measured at a clinical visit. FIG. 3A, shows, for each sample, the measured height of the child associated with that sample, and the predicted height of the child based on models trained on data derived for tooth topography. Predicted values of height were highly correlated (r=0.98) with measured values of height, indicating high accuracy in the predictive model. FIG. 3B shows, for each sample, the measured weight of the child associated with that sample and the predicted weight of the child based on a model trained on features derived from that samples tooth topography. Predicted values of BMI child weight were highly correlated (r=0.92) with measured values of weight, indicating high accuracy in the predictive model.

### Example 2: Tooth Topographic Data as a Predictor for Measured Height and Weight of a Child

A sample set of 150 subjects collected in another population-based cohort study, ex-vivo teeth were analyzed with the bench-top system with TPE gel cartridges. The ex-vivo teeth samples were obtained from one to five years from the moment of shedding. Each tooth was then scanned with the bench-top system, described elsewhere herein, to generate a 3-D topographic dataset of the top surface of the tooth. The 3-D topographic datasets were then analyzed, as described elsewhere herein, to generate one or more features of the 3-D topographic dataset. One of such features calculated from the 3-D topographic dataset included a linear profile of the 3-D topographic dataset measured from a tooth's incisal edge to the cervical end of the crown on the labial/buccal side of the crown. Additional features descriptive of tooth topography were derived as described and used in the construction of a model predictive of anthropometric features at 6 years of age, including height and body mass index (BMI) measured at a clinical visit. FIG. 4A shows, for each sample, the measured height of the child associated with that sample, and the predicted height of the child based on models trained on data derived for tooth topography. Predicted values of height were highly correlated (r=0.97) with measured values of height, indicating high accuracy in the predictive model. FIG. 4B shows, for each sample, the measured BMI of the child associated with that sample and the predicted BMI based on a model trained on features derived from that samples tooth topography. Predicted values of BMI were highly correlated (r=0.99) with measured values of height, indicating high accuracy in the predictive model.

### Example 3: Tooth Topographic Data as a Predictor for IQ and Child Cognitive Composite Score

A sample set of 150 subjects collected in a population-based cohort Study 2 (seen in Example 2) ex-vivo teeth were analyzed with the bench-top system with TPE gel cartridges. The ex-vivo teeth samples were obtained from one to five years from the moment of shedding. Each tooth was then scanned with the bench-top system, described elsewhere herein, to generate a 3-D topographic dataset of the top surface of the tooth. The 3-D topographic datasets were then analyzed, as described elsewhere herein, to generate one or more features of the 3-D topographic dataset. One of such features calculated from the 3-D topographic dataset included a linear profile of the 3-D topographic dataset measured from a tooth's incisal edge to the cervical end of the crown on the labial/buccal side of the crown. Additional features descriptive of tooth topography were also derived as described and used in the construction of a model predictive of IQ and cognitive development. These phenotypic characteristics were measured at 6 years of age using standard psychological assays, specifically, the Weschler Intelligence Scale for Children (WISC) and the Behavior Assessment System for Children (BASC), respectively. FIG. 5A shows, for each sample, the measured IQ of the child associated with that sample, and the predicted IQ of the child based on models trained on data derived for tooth topography. Predicted and measured IQ scores were highly correlated (r=0.99), indicating high accuracy in the predictive model. FIG. 5B shows the measured cognitive composite score for each child, and the predicted cognitive composite score based on the predictive model. The correlation between measured and predictive cognitive composite scores was high (r=0.98), indicating accurate performance of the model.

### Example 4: Tooth Topographic Data as a Predictor for IQ and Child Cognitive Composite Score

In a sample set of 127 subjects collected in population-based cohort (subset of subjects of Example 2), a parallel analysis was conducted with standard mass spectrometry methods to determine the concentration of various chemical elements, including zinc (Zn), lead (Pb), copper (Cu), arsenic (As), manganese (Mn), cadmium (Cd), magnesium (Mg), calcium (Ca), and chromium (Cr). Features descriptive of tooth topography (prior to chemical analysis) were then derived as described and used in the construction of a model predictive of elemental biomarker concentrations. For each element the metal concentration measured in each tooth with mass spectrometry was plotted against the concentration predicted for that tooth based on models trained on features derived from analysis of tooth surface topography (FIGS. 7A-7I). Model performance was highly accurate, with correlations between measured and predicted elemental concentrations varying from 0.87-0.96. More specifically, for Zn (FIG. 7A) model performance yielded a correlation between measured and predicted values of r=0.96. For Pb (FIG. 7B), model performance yielded a correlation between measured and predicted values of r=0.91. For Cu (FIG. 7C) model performance yielded a correlation between measured and predicted values of r=0.96. For As (FIG. 7D) model performance yielded a correlation between measured and predicted values of r=0.87. For Mn (FIG. 7E), model performance yielded a correlation between measured and predicted values of r=0.91. For Cd (FIG. 7F), model performance yielded a correlation between measured and predicted values of r=0.90. For Mg (FIG. 7G), model performance yielded a correlation between measured and predicted values of r=0.91. For Ca (FIG. 7H), model performance yielded a correlation between measured and predicted values of r=0.91. For Cr (FIG. 7I), model performance yielded a correlation between measured and predicted values of r=0.92.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining the topography of a surface of a biological sample, comprising:
(a) receiving a biological sample;
(b) mounting the biological sample into a fixture;
(c) contacting the biological sample with a compressible body optically coupled to at least one light source and at least one sensor, wherein the at least one light source generates a multi-dimensional dataset of the surface of the biological sample detected by the at least one sensor; and
(d) determining a surface profile of the biological sample from the multi-dimensional dataset.

2. The method of claim 1, wherein the biological sample comprises an ex-vivo or in-vivo tooth.

3. The method of claim 1, wherein the compressible body comprises a gel cartridge.

4. The method of claim 1, wherein the fixture comprises putty, a mechanical mount, adhesive compound, or any combination thereof.

5. The method of claim 1, further comprising calibrating the compressible body on standard glass, ball grid array, groove targets, or any combination thereof.

6. The method of claim 1, wherein the at least one light source comprises at least one coherent laser, incoherent laser, pulsed laser, light emitting diode, super-luminescent diode, or any combination thereof.

7. The method of claim 1, further comprising cleaning the biological sample with water.

8. The method of claim 1, wherein the compressible body spatial position is releasably lockable.

9. The method of claim 1, wherein the at least one light source and the at least one sensor are electrically in communication with a processor via a button, switch, trigger, or any combination thereof configured to initiate illumination of the biological sample and detection of the multi-dimensional dataset of the surface.

10. The method of claim 1, wherein the multi-dimensional dataset comprises at least 1 dimension.

11. The method of claim 2, wherein the surface profile is measured from the incisal edge to the cervical end of the crown of the ex-vivo or in-vivo tooth.

12. A method for determining the topography of a surface of a biological sample, comprising:
(a) receiving a biological sample;
(b) mounting the biological sample into a fixture;
(c) illuminating the biological sample with at least one light source and at least one sensor, wherein the at least one light source generates a multi-dimensional dataset of the surface of the biological sample detected by the at least one sensor; and
(d) determining a surface profile of the biological sample from the multi-dimensional dataset.

13. The method of claim 12, wherein the biological sample comprises ex-vivo or in-vivo tooth.

14. The method of claim 12, wherein the fixture comprises putty, a mechanical mount, stabilizer, adhesive compound, or any combination thereof.

15. The method of claim 12, wherein the at least one light source comprises at least one coherent laser, incoherent laser, pulsed laser, light emitting diode, super-luminescent diode, or any combination thereof.

16. The method of claim 12, further comprising cleaning the biological sample with water.

17. The method of claim 12, wherein the at least one light source and the at least one sensor are electrically in communication with a processor via a button, switch, trigger, or any combination thereof configured to initiate illumination of the biological sample and detection of the multi-dimensional dataset of the surface.

18. The method of claim 12, wherein the multi-dimensional dataset comprises at least 1 dimension.

19. The method of claim 13, wherein the surface profile is measured from an incisal edge to a cervical end of a crown of the ex-vivo or in-vivo tooth.

20. A system for determining the topography of a surface of a biological sample, comprising:
(a) a fixture mechanically configured to constrain a three-dimensional position of a biological sample;
(b) a compressible body optically coupled to at least one light source and at least one sensor, wherein the at least one light source generates a multi-dimensional dataset of a surface of the biological sample detected by the at least one sensor; and
(c) a processor electrically coupled to the at least one light source and the at least one sensor, wherein the processor comprises a set of programmed instructions stored on a non-transitory storage medium configured to cause the processor to determine a topography of the surface of the biological sample from the multi-dimensional dataset.
